(19) United States
(12) Patent Application Publication
(10) Pub. No.: US 2026/0241000 A1
(43) Pub. Date: Aug. 20, 2026

(54) CD79B HUMANIZED ANTIBODY-BASED CHIMERIC ANTIGEN RECEPTOR AND USE THEREOF

(71) Applicant: BEIJING MEIKANG GENO-IMMUNE BIOTECHNOLOGY CO., LTD., Beijing (CN)

(72) Inventor: Lung-ji Chang, Beijing (CN)

(21) Appl. No.: 18/872,125

(22) PCT Filed: May 29, 2023
(86) PCT No.: PCT/CN2023/096720

§ 371 Date: Dec. 5, 2024

(30) Foreign Application Priority Data
Jun. 9, 2022 (CN) 202210649400.X

(51) Int. Cl. A61K 40/42; A61K 40/11; A61K 40/31; A61P 35/00; C07K 14/705; C07K 16/28; C12N 5/0783; C12N 9/64; C12N 15/86

(52) U.S. Cl. CPC A61K 40/421; A61K 40/11; A61K 40/31; A61P 35/00; C07K 14/7051; C07K 14/70521; C07K 14/70578; C07K 16/2803; C12N 5/0636; C12N 9/6472; C12N 15/86; C12Y 304/22062; C07K 2317/24; C07K 2319/02; C07K 2319/03; C12N 2510/00; C12N 2740/15043; C12N 2740/15052

(57) ABSTRACT

Provided are a CD79b humanized antibody-based chimeric antigen receptor and use thereof. The present disclosure relates to a CD79b humanized antibody-based chimeric antigen receptor, which includes an antigen binding domain, a transmembrane domain, a co-stimulatory signaling domain, a CD3q signaling domain, and a self-destructing domain, which are sequentially connected in tandem, wherein the antigen binding domain binds to a tumor surface antigen CD79b and is a humanized scFv targeting the tumor surface antigen CD79b, and the self-destructing domain is a cysteine protease 9 domain. When the chimeric antigen receptor is applied to patients with tumors expressing the tumor-specific target CD79b, few clinical side effects are generated, the safety is high, tumor cells can be effectively killed, and the presence of CAR-T in the patients can be monitored for a long time, effectively prolonging the overall survival rate of patients.

### TECHNICAL FIELD

The present disclosure relates to the field of cellular immunotherapy of tumors, in particular, to a CD79b humanized antibody-based chimeric antigen receptor and use thereof, and specifically, to a method for constructing chimeric antigen receptor T (CAR-T) cells based on a tumor-specific target CD79b and use thereof in anti-tumor therapy.

### BACKGROUND

With the development of tumor immunology theories and clinical techniques, chimeric antigen receptor T cell (CAR-T) immunotherapy has become one of the most promising tumor immunotherapies. Generally, a chimeric antigen receptor (CAR) is composed of a tumor-associated antigen binding region, an extracellular hinge region, a transmembrane domain, and an intracellular cell signaling domain. Generally, the CAR contains a single-chain variable fragment (scFv) region of an antibody or a domain specifically binding to a tumor-associated antigen (TAA), which is coupled to a cytoplasmic domain of a T cell signaling molecule through a hinge and a transmembrane domain. The most common lymphocyte activation moiety includes a T cell co-stimulatory domain in tandem with a moiety (for example, CD3ζ) triggering T-cell effector function. CAR-mediated adoptive immunotherapy allows CAR-grafted T cells to directly recognize TAAs on target tumor cells in a non-human leukocyte antigen (HLA)-restricted manner.

The B-cell lymphomas are solid tumors affecting B cells. B-cell lymphomas include both Hodgkin lymphomas and non-Hodgkin lymphomas. There are numerous types of B-cell lymphomas, and classic Hodgkin lymphoma and nodular lymphocyte predominant Hodgkin lymphoma are now considered tumors derived from B cells. Diffuse large B-cell lymphoma, follicular lymphoma, mucosa-associated lymphatic tissue (MALT) lymphoma, small lymphocytic lymphoma/chronic lymphocytic leukemia and mantle cell lymphoma (MCL) are five most common types of B-cell non-Hodgkin lymphoma and account for three out of four patients with non-Hodgkin lymphoma.

In addition, B-cell lymphomas are divided into indolent lymphomas and aggressive lymphomas depending on differences in clinical manifestations. Indolent lymphomas are generally slow-developing, are kept under control with the stabilized state of disease and long-term survival of many years, but cannot be cured. Aggressive lymphomas usually require intensive treatments, with some having a good prospect for a permanent cure. The prognosis and treatment of B-cell lymphomas depend on the specific type of lymphoma as well as the stage and grade. One method for treating patients with B-cell lymphomas is to perform gene modification on T cells through CAR expression to target antigens expressed on tumor cells. The CAR is an antigen receptor designed for recognizing antigens on the surface of cells in a HLA-independent manner. Attempts to treat these patients with CAR-expressing genetically modified cells have achieved promising success.

CD79b, as a moiety of the B-cell receptor (BCR) signaling complex, is a key cell surface receptor for the successful development and maintenance of mature B cells. CD79b is generally expressed exclusively in B-cell lines and remains highly expressed in most non-Hodgkin lymphoma subtypes, so it is one of the ideal tumor antigens for targeting B-cell-associated tumors in immunotherapies. At present, among immunotherapies of B-cell tumors, the antibody therapy against CD19 has been developed maturely. Such a method has achieved initial success in clinical practice. However, the problems of the antibody therapy are that after the antibody is administrated, the antibody exists in the peripheral blood and fails to accurately enter the tumor tissue or the site of minimal residuals of tumors and the antibody cannot be present in vivo for a long time after administration. Moreover, such an anti-CD19 antibody is a human-mouse chimeric antibody structure and may be resistant to the human body, increasing the difficulty of retreatment.

Therefore, fully humanized chimeric antigen receptors targeting B-cell surface antigens except for CD19 are developed. For example, CAR-T cells of CD79b not only have the advantages of targeted therapy of the antibody, but also can accurately enter the tumor tissue and be present in vivo for a long time because of the characteristics of T cells and the high expression of tumor surface antigens, which will definitely provide more effective therapeutic options for relapsed and refractory B-cell tumors.

### SUMMARY

The number of targeted antigens in the tumor treatment by CAR-T technology at present is still small and the tumor microenvironment affects the therapeutic effect of CAR-T technology. The objective of the present disclosure is to provide a CD79b humanized antibody-based chimeric antigen receptor and use thereof. The chimeric antigen receptor (4SCAR-79b) prepared by the present disclosure optimizes and modifies CAR-T cells modified by humanized CD79b antibody genes, thereby improving the immune long-term efficacy and safety of antigen targets and enhancing the therapeutic effect of CAR-T cells.

To achieve the objective, the present disclosure adopts the technical solutions described below.

In a first aspect, the present disclosure provides a CD79b humanized antibody-based chimeric antigen receptor (4SCAR-79b), which includes an antigen binding domain, a transmembrane domain, a co-stimulatory signaling domain, a CD3ζ signaling domain, and a self-destructing domain, which are sequentially connected in tandem;
the antigen binding domain is a single-chain variable fragment (scFv) against a tumor surface antigen CD79b, and the amino acid sequence of the scFv against the tumor surface antigen CD79b is selected from:
an amino acid sequence shown in SEQ ID NO: 1; or an amino acid sequence which is formed by amino acid substitutions, additions or deletions to the amino acid sequence shown in SEQ ID NO: 1, where the amino acid sequence specifically binds to the chimeric antigen receptor and has functions of binding to CD79b and inducing T cell signaling.

In the present disclosure, the amino acid substitutions may be one or more.

In the present disclosure, the antigen binding domain binds to a tumor surface antigen that is CD79b. By modifying the scFv and the specially designed gene structure of the CAR, the genetically modified CAR-T cells can specifically bind to the tumor surface antigen, and relatively moderate signal stimulation is obtained, thereby exerting an effective killing effect. Meanwhile, the immune factors are slowly released, thereby reducing the risk of cytokine storms. The chimeric antigen receptor and the tumor antigen herein have a better effect and higher safety than other chimeric antigen receptors and other tumor antigens.

The amino acid sequence (SEQ ID NO: 1) of the scFv against the tumor surface antigen CD79b is as follows:

QVQLVESGPGVVQPGRSLRISCAVSGFSLSNYAVSWVRQPPGKGLEWLGIIYIETGTT
WYANWAKGRLTISKDNSKNTVYLQMNSLRAEDTAMYYCASEPYEPYDDSNIYYGMDP
WGQGTLVTVSSGSTSGSGKPGSSEGSTKGEIVMTQTPATLSVSAGERVTITCQASQSVVS
GNYLAWYQQKPGQAPRLLIYSASTLASGVPARFSGSGYGTEFTFTISSVQSEDFAVYFCL
GEFSCSSHDCNAFGQGTKLEIK.

In the present disclosure, the CAR signal structure targeting the tumor surface antigen CD79b is specifically modified, and modification can also be quickly performed for different CD79b scFvs so that the modified 4SCAR-79b shows a stronger immunostimulatory force.

Preferably, the amino acid sequence which is formed by amino acid substitutions, additions or deletions to the amino acid sequence shown in SEQ ID NO: 1 has at least 90%, for example, 90%, 91%, 92%, 93%, 94%, 95%, 96%, 97%, 98%, 99% or 100%, identity to the amino acid sequence shown in SEQ ID NO: 1 and preferably, is an amino acid sequence having at least 95% identity.

In the present disclosure, the modified amino acid sequence can still specifically bind to the chimeric antigen receptor and has the functions of binding to CD79b and inducing T cell signaling.

Preferably, the transmembrane domain is a CD28 transmembrane domain and/or a CD8a transmembrane domain.

In the present disclosure, the CD28 transmembrane domain includes a CD28 extracellular signaling domain and a CD28 cell membrane signaling domain.

In some embodiments, the transmembrane domain may be selected or modified through amino acid substitutions.

Preferably, the co-stimulatory signaling domain is a combination of a CD28 signaling domain (also specifically referred to herein as a CD28 intracellular signaling domain) and a CD27 signaling domain (also specifically referred to herein as a CD27 intracellular signaling domain).

In the present disclosure, the CD28 extracellular signaling domain, the CD28 cell membrane signaling domain and the CD28 intracellular signaling domain together form a CD28 full signaling domain. The arrangement of the CD28 full signaling domain and the CD27 signaling domain can be adjusted as required by those skilled in the art. The different arrangements of the CD28 full signaling domain and the CD27 signaling domain have no effect on the chimeric antigen receptor, and the sequential combination of CD28-CD27 is adopted in the present application.

Herein, the CD28-CD27 refers to a CD28 full signaling domain and a CD27 signaling domain.

Preferably, the amino acid sequence of the CD28-CD27 is shown in SEQ ID NO: 2.

The specific sequence of SEQ ID NO: 2 is as follows:

IEVMYPPPYLDNEKSNGTIIHVKGKHLCPSPLFPGPSKPFWVLVVVGGVLACYSLLV
TVAFIIFWVRSKRSRLLHSDYMNMTPRRPGPTRKHYQPYAPPRDFAAYRSASGGGGSGG
GGSQRRKYRSNKGESPVEPAEPCHYSCPREEEGSTIPIQEDYRKPEPACSP.

In the present disclosure, the co-stimulatory signaling domain further includes a linker sequence, where the linker sequence is a repeated sequence of two or more GGGGS (SEQ ID NO: 11) or related region linker sequences.

In the present disclosure, the CD28 full signaling domain includes a CD28 extracellular signaling domain, a CD28 cell membrane signaling domain and a CD28 intracellular signaling domain.

The amino acid sequence of the CD28 extracellular signaling domain is shown in SEQ ID NO: 3 which is specifically as follows:

IEVMYPPPYLDNEKSNGTIIHVKGKHLCPSPLFPGPSKP.

The amino acid sequence of the CD28 cell membrane signaling domain is shown in SEQ ID NO: 4 which is specifically as follows:

FWVLVVVGGVLACYSLLVTVAFIIFWV.

The amino acid sequence of the CD28 intracellular signaling domain is shown in SEQ ID NO: 5 which is specifically as follows:

RSKRSRLLHSDYMNMTPRRPGPTRKHYQPYAPPRDFAAYRS.

The CD27 signaling domain includes a CD27 intracellular signaling domain, and the amino acid sequence of the CD27 intracellular signaling domain is shown in SEQ ID NO: 6 which is specifically as follows:

QRRKYRSNKGESPVEPAEPCHYSCPREEEGSTIPIQEDYRKPEPACSP.

Preferably, the self-destructing domain includes a cysteine protease 9 domain.

Preferably, the amino acid sequence of the cysteine protease 9 domain is shown in SEQ ID NO: 7, and the amino acid sequence (SEQ ID NO: 7) of the cysteine protease 9 domain is as follows:

MGVQVETISPGDGRTFPKRGQTCVVHYTGMLEDGKKVDSSRDRNKPFKFMLGKQ
EVIRGWEEGVAQMSVGQRAKLTISPDYAYGATGHPGIIPPHATLVFDVELLKLEGGGGSG
GGGSGAMVGALESLRGNADLAYILSMEPCGHCLIINNVNFCRESGLRTRTGSNIDCEKLR
RRFSSLHFMVEVKGDLTAKKMVLALLELARQDHGALDCCVVVILSHGCQASHLQFPGA
VYGTDGCPVSVEKIVNIFNGTSCPSLGGKPKLFFIQACGGEQKDHGFEVASTSPEDESPGS
NPEPDATPFQEGLRTFDQLDAISSLPTPSDIFVSYSTFPGFVSWRDPKSGSWYVETLDDIFE
QWAHSEDLQSLLLRVANAVSVKGIYKQMPGCFNFLRKKLFFKTSAS.

Preferably, the self-destructing domain is connected in tandem with the CD3ζ signaling domain through a 2A sequence.

In the present disclosure, the 2A sequence can cleave the protein expressed by the self-destructing domain and the protein of the chimeric antigen receptor, enabling the chimeric antigen receptor to function. With an activator injected, the self-destructing domain is activated, causing the chimeric antigen receptor to become inactive.

In the present disclosure, the chimeric antigen receptor further includes a signal peptide. The signal peptide may be any signal peptide capable of directing the transfer of the chimeric antigen receptor across transmembrane, and those skilled in the art may select a conventional signal peptide in the art as required. The signal peptide is a Secretory signal peptide, and the amino acid sequence of the Secretory signal peptide includes SEQ ID NO: 8 or SEQ ID NO: 9.

SEQ ID NO: 8 is
MLLLVTSLLLCELPHPAFLLIP.
SEQ ID NO: 9 is
MALPVTALLLPLALLLHAARP.

In the present disclosure, the chimeric antigen receptor may further include a hinge region. The hinge region may be selected by those skilled in the art according to actual conditions and is not particularly limited herein, and the presence of the hinge region has no effect on the performance of the chimeric antigen receptor of the present disclosure.

In the present disclosure, the chimeric antigen receptor may further include a promoter. The promoter may be EF1a or any highly expressed promoter. The promoter may be selected by those skilled in the art according to actual conditions and is not particularly limited herein, and the presence of the promoter has no effect on the performance of the chimeric antigen receptor of the present disclosure.

Preferably, the chimeric antigen receptor includes a signal peptide, an antigen binding domain, a transmembrane domain, a co-stimulatory signaling domain, a CD3ζ signaling domain, a 2A sequence, and a self-destructing domain, which are sequentially connected in tandem.

Preferably, the chimeric antigen receptor is formed by connecting in tandem a Secretory signal peptide, a CD79b antigen binding domain, a CD28 transmembrane domain and/or a CD8a transmembrane domain, a CD28 signaling domain, a CD27 signaling domain, a CD3ζ signaling domain, a 2A sequence, and a cysteine protease 9 domain.

Preferably, the amino acid sequence of the chimeric antigen receptor is shown in SEQ ID NO: 10.

In the present disclosure, the composition of the chimeric antigen receptor is Secretory signal-CD79b scFv-CD28-CD27-CD3ζ-2A-FBKP.Casp9. The Secretory signal-CD79b scFv-CD28-CD27-CD3ζ-2A-FBKP.Casp9 is designated as the 4SCAR-79b chimeric antigen receptor. The 4SCAR-79b chimeric antigen receptor is formed by connecting in tandem a Secretory signal peptide, a humanized single-chain CD79b antigen binding domain, a CD28 transmembrane domain, a CD28 signaling domain, a CD27 signaling domain, a CD3ζ signaling domain, a 2A sequence, and a cysteine protease 9 domain which are specifically arranged as follows:

Secretory signal-CD79b scFv-CD28-CD27-CD3ζ-2A-FBKP.Casp9.

In the present disclosure, the amino acid sequence of the chimeric antigen receptor (4SCAR-79b) Secretory signal-CD79b scFv-CD28-CD27-CD3ζ-2A-FBKP.Casp9 is shown in SEQ ID NO: 10.

SEQ ID NO: 10 is as follows:

MLLLVTSLLLCELPHPAFLLIPQVQLVESGPGVVQPGRSLRISCAVSGFSLSNYAVSW
VRQPPGKGLEWLGIIYIETGTTWYANWAKGRLTISKDNSKNTVYLQMNSLRAEDTAMY
YCASEPYEPYDDSNIYYGMDPWGQGTLVTVSSGSTSGSGKPGSSEGSTKGEIVMTQTPAT
LSVSAGERVTITCQASQSVVSGNYLAWYQQKPGQAPRLLIYSASTLASGVPARFSGSGYG
TEFTFTISSVQSEDFAVYFCLGEFSCSSHDCNAFGQGTKLEIKAAAIEVMYPPPYLDNEKS
NGTIIHVKGKHLCPSPLFPGPSKPFWVLVVVGGVLACYSLLVTVAFIIFWVRSKRSRLLHS
DYMNMTPRRPGPTRKHYQPYAPPRDFAAYRSASGGGGSGGGGSQRRKYRSNKGESPVE
PAEPCHYSCPREEEGSTIPIQEDYRKPEPACSPGGGGSGGGGSRVKFSRSADAPAYQQGQ
NQLYNELNLGRREEYDVLDKRRGRDPEMGGKPRRKNPQEGLYNELQKDKMAEAYSEIG
MKGERRRGKGHDGLYQGLSTATKDTYDALHMQALPPRTSGSGATNFSLLKQAGDVEEN
PGPMGVQVETISPGDGRTFPKRGQTCVVHYTGMLEDGKKVDSSRDRNKPFKFMLGKQE
VIRGWEEGVAQMSVGQRAKLTISPDYAYGATGHPGIIPPHATLVFDVELLKLEGGGGSGG
GGSGAMVGALESLRGNADLAYILSMEPCGHCLIINNVNFCRESGLRTRTGSNIDCEKLRR
RFSSLHFMVEVKGDLTAKKMVLALLELARQDHGALDCCVVVILSHGCQASHLQFPGAV
YGTDGCPVSVEKIVNIFNGTSCPSLGGKPKLFFIQACGGEQKDHGFEVASTSPEDESPGSN
PEPDATPFQEGLRTFDQLDAISSLPTPSDIFVSYSTFPGFVSWRDPKSGSWYVETLDDIFEQ
WAHSEDLQSLLLRVANAVSVKGIYKQMPGCFNFLRKKLFFKTSAS.

In a second aspect, the present disclosure provides a nucleic acid molecule. The nucleic acid molecule encodes the CD79b humanized antibody-based chimeric antigen receptor described in the first aspect.

In a third aspect, the present disclosure provides a viral vector. The viral vector includes at least one copy of the nucleic acid molecule described in the second aspect.

Preferably, the viral vector is a lentiviral vector and/or a retroviral vector and preferably, is a lentiviral vector.

In a fourth aspect, the present disclosure provides a recombinant lentivirus. The recombinant lentivirus is prepared by a preparation method including the following step: co-transducing the viral vector described in the third aspect with packaging helper plasmids pNHP and pHEF-VSVG into a mammalian cell to obtain the recombinant lentivirus.

Preferably, the mammalian cell includes a 293 cell, a 293 T cell or a TE671 cell.

In a fifth aspect, the present disclosure provides a chimeric antigen receptor T cell. The chimeric antigen receptor T cell is prepared by a preparation method including the following step: transducing the recombinant lentivirus described in the fourth aspect into a T cell for expression to obtain the chimeric antigen receptor T cell.

In the present disclosure, the T cell has good targeting and killing effects, is capable of releasing a low dosage of immune factors, and has properties of low toxicity and a high immune killing response.

In a sixth aspect, the present disclosure provides a composition. The composition includes any one or a combination of at least two of the CD79b humanized antibody-based chimeric antigen receptor described in the first aspect, the recombinant lentivirus described in the fourth aspect or the chimeric antigen receptor T cell described in the fifth aspect.

In a seventh aspect, the present disclosure provides a use of any one or a combination of at least two of the CD79b humanized antibody-based chimeric antigen receptor described in the first aspect, the recombinant lentivirus described in the fourth aspect, the T cell described in the fifth aspect or the composition described in the sixth aspect in the preparation of a tumor treatment drug.

Preferably, the tumor is a tumor disease in which a CD79b specific antigen is expressed.

Preferably, the tumor disease in which the CD79b specific antigen is expressed is a B-cell tumor.

Compared with the existing art, the present disclosure has the following beneficial effects.
(1) The chimeric antigen receptor of the present disclosure is obtained by specific genetic modification of the T cell intracellular co-stimulatory signaling domain of the chimeric antigen receptor targeting the tumor surface antigen CD79b. The modified chimeric antigen receptor has a better response effect after specifically binding to the CD79b so that the CAR-T cell has a stronger immune response to the tumor.
(2) After being applied to the human body, the chimeric antigen receptor T cell of the present disclosure has a higher safety than other chimeric antigen receptor T cells targeting the CD79b, and even if adverse effects occur, the chimeric antigen receptor T cell can be removed using a drug that induces apoptosis of CAR-T cells due to the presence of the signal inducing the apoptosis mechanism.
(3) With the 4SCAR-79b chimeric antigen receptor of the present disclosure, the presence of CAR-T can be monitored in vivo for a long time after the CAR-T cells are infused, proving that the 4SCAR-79b chimeric antigen receptor has long-term efficacy and can provide long-term relief for patients.

### BRIEF DESCRIPTION OF DRAWINGS

FIG. 1 is a schematic diagram of the mechanism of chimeric antigen receptor T cells in Example 1;

FIG. 2 is a structure diagram of a 4SCAR-79b chimeric antigen receptor in Example 1;

FIG. 3 is a plasmid map of the backbone vector pTYF of a lentiviral vector in Example 1;

FIG. 4A is images showing in vitro killing of CD79b-positive tumor cell strains by different types of T cells at 24 h and 48 h in Example 5 (with a magnification factor of 50×);

FIG. 4B is an image showing statistical results of remaining target cells quantified by flow cytometry after the killing of CD79b-positive tumor cell strains by different types of T cells at 24 h and 48 h in Example 5;

FIG. 4C is an image showing statistical results of target cell death percentages after the killing of CD79b-positive tumor cell strains by different types of T cells at 24 h in Example 5;

FIG. 5 is a flowchart of 4SCAR-79b-CAR-T treatment for a B-cell tumor in Example 6;

FIG. 6 is an image showing immunohistochemical staining results of tumor sections of a patient with DLBCL in Example 6 (with a magnification factor of 20×);

FIG. 7 is a graph of in vivo CAR copy numbers detected in the DLBCL patient infused with 4SCAR-79b-CAR-T in Example 6; and

FIG. 8 is images showing the change of the tumor on the right face of the DLBCL patient before and after infusion with 4SCAR-79b-CAR-T in Example 6.

### DETAILED DESCRIPTION

Technical solutions of the present disclosure are further described below through examples. It is to be understood by those skilled in the art that the examples described below are used for a better understanding of the present disclosure and are not to be construed as specific limitations to the present disclosure.

Experiments without specific techniques or conditions specified in the examples are conducted according to techniques or conditions described in the literature in the art or a product specification. The reagents or instruments used herein without manufacturers specified are conventional products commercially available from proper channels.

### Example 1: Construction of a Chimeric Antigen Receptor

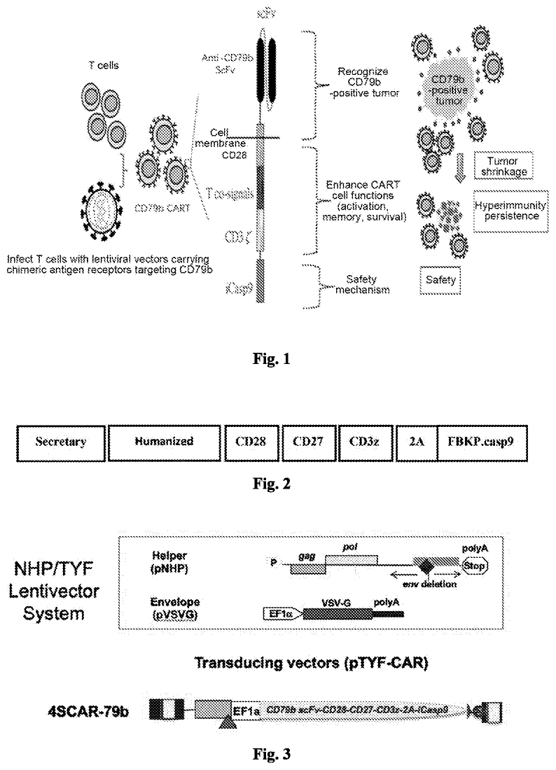

The schematic diagram of the mechanism of chimeric antigen receptor T cells is shown in FIG. 1. A Secretory signal peptide, a humanized single-chain CD79b scFv antigen binding domain, a CD28 extracellular signaling domain, a CD28 cell membrane signaling domain, a CD28 signaling domain, a CD27 signaling domain, a CD3ζ signaling domain, a 2A sequence, and a cysteine protease 9 domain were synthesized by whole gene synthesis. The obtained chimeric antigen receptor Secretory signal-CD79b scFv-CD28-CD27-CD3ζ-2A-FBKP.Casp9 was designated as 4SCAR-79b chimeric antigen receptor, and the structure diagram of the 4SCAR-79b chimeric antigen receptor is shown in FIG. 2.

The amino acid sequence SEQ ID NO: 10 of the chimeric antigen receptor was as follows:

MLLLVTSLLLCELPHPAFLLIPQVQLVESGPGVVQPGRSLRISCAVSGFSLSNYAVSW
VRQPPGKGLEWLGIIYIETGTTWYANWAKGRLTISKDNSKNTVYLQMNSLRAEDTAMY
YCASEPYEPYDDSNIYYGMDPWGQGTLVTVSSGSTSGSGKPGSSEGSTKGEIVMTQTPAT
LSVSAGERVTITCQASQSVVSGNYLAWYQQKPGQAPRLLIYSASTLASGVPARFSGSGYG
TEFTFTISSVQSEDFAVYFCLGEFSCSSHDCNAFGQGTKLEIKAAAIEVMYPPPYLDNEKS
NGTIIHVKGKHLCPSPLFPGPSKPFWVLVVVGGVLACYSLLVTVAFIIFWVRSKRSRLLHS
DYMNMTPRRPGPTRKHYQPYAPPRDFAAYRSASGGGGSGGGGSQRRKYRSNKGESPVE
PAEPCHYSCPREEEGSTIPIQEDYRKPEPACSPGGGGSGGGGSRVKFSRSADAPAYQQGQ
NQLYNELNLGRREEYDVLDKRRGRDPEMGGKPRRKNPQEGLYNELQKDKMAEAYSEIG
MKGERRRGKGHDGLYQGLSTATKDTYDALHMQALPPRTSGSGATNFSLLKQAGDVEEN
PGPMGVQVETISPGDGRTFPKRGQTCVVHYTGMLEDGKKVDSSRDRNKPFKFMLGKQE
VIRGWEEGVAQMSVGQRAKLTISPDYAYGATGHPGIIPPHATLVEDVELLKLEGGGGSGG
GGSGAMVGALESLRGNADLAYILSMEPCGHCLIINNVNFCRESGLRTRTGSNIDCEKLRR
RFSSLHFMVEVKGDLTAKKMVLALLELARQDHGALDCCVVVILSHGCQASHLQFPGAV
YGTDGCPVSVEKIVNIFNGTSCPSLGGKPKLFFIQACGGEQKDHGFEVASTSPEDESPGSN
PEPDATPFQEGLRTFDQLDAISSLPTPSDIFVSYSTFPGFVSWRDPKSGSWYVETLDDIFEQ
WAHSEDLQSLLLRVANAVSVKGIYKQMPGCFNFLRKKLFFKTSAS.

A nucleotide sequence encoding the 4SCAR-79b chimeric antigen receptor was ligated with the backbone vector pTYF of a lentiviral vector to obtain a pTYF DNA vector. The plasmid map of the backbone vector pTYF of the lentiviral vector is shown in FIG. 3.

### Example 2: Lentivirus Packaging

(1) 293T cells were incubated for 17-18 h.
(2) Fresh DMEM containing 10% FBS was added.
(3) DMEM was added to a sterile centrifuge tube together with the following reagents: helper DNA mix (pNHP, pHEF-VSV-G, and GFP reporter plasmid) and pTYF DNA vectors, swirled and oscillated.
(4) Superfect or a similar transduction gene material was added to the centrifuge tube, blown 5 times, and allowed to stand for 7-10 min at room temperature.
(5) The DNA-Superfect mixture solution in the centrifuge tube was added dropwise to each culture well and swirled evenly.
(6) The system was incubated for 4-5 h at 37° C. in a CO₂ incubator.
(7) Cells were washed with 1.5 mL of fresh medium, and a medium was added to continue the culture.
(8) The incubated cells were placed back in the incubator and incubated overnight, and the GFP expression was observed the following morning with a fluorescence microscope to assess the transfection efficiency.

### Example 3: Purification and Concentration of Lentiviruses

### 1) Virus Purification

Cell debris was removed through centrifugation (at 1000 g) to obtain the virus supernatant. The virus supernatant was filtered by a low protein binding filter, and the viruses were divided into small portions and stored at −80° C.

Generally, the transduced cells may produce lentiviral vectors with a titer of greater than 107 transduction units per milliliter of the culture medium.

2) Concentration of Lentiviral Vectors with a Filter
(1) The concentration tube was washed with sterile PBS in a biosafety cabinet.
(2) The virus supernatant was added to the tube and centrifuged at 2500 g until the virus volume was reduced by a factor of 50.
(3) The tube was shaken and centrifuged at 400 g, the concentrated viruses were collected into a collection cup, and finally the viruses in all tubes were pooled into one centrifuge tube. After concentration, lentiviral vectors with a titer of greater than 10° transduction units may be produced.

### Example 4: Preparation of 4SCAR-79b-CAR-T Cells

The activated T cells were inoculated into a medium, and polybrene was added, where the medium contained T cell growth factors comprising IL-2. The concentrated CAR gene lentiviruses were added, centrifuged for 100 min at a centrifugal speed of 100 g and incubated overnight. A medium was added and incubated for 4 days, the cells were harvested and counted, and the target cell killing and safety detection was performed. The cells were incubated for another 1-2 days and then infused to a patient.

The 4SCAR-79b-CAR-T cells can effectively shrink the tumor and are safe, which would be verified by in vitro and in vivo assays, respectively.

### Example 5: In Vitro Killing Assay of 4SCAR-79b-CAR-T Cells

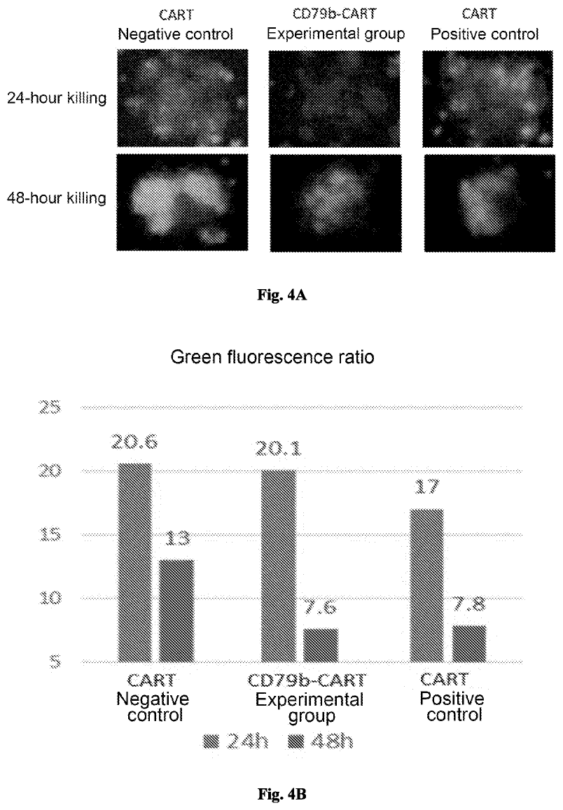

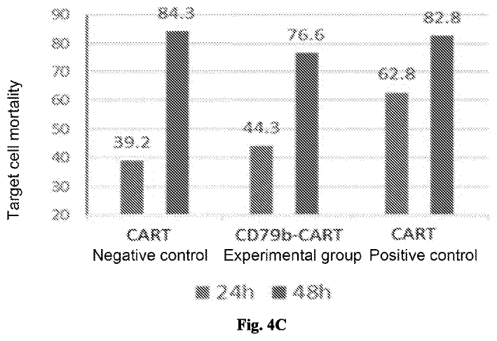

(1) A CD79b-positive tumor cell strain was transduced with green fluorescent proteins (GFPs) via lentiviral vectors to make the cell strain stably express GFPs and serve as marker target cells.
(2) Non-specific control T cells (T cells without CAR transgenes, or negative control CAR-T cells that target an antigen not expressed by target cells, such as CD33-CART) or positive control CAR-T cells targeting an antigen different from the CD79b scFv (control CAR-T cells that target another antigen expressed by target cells, such as CD19-CART) were co-incubated with the above tumor in a 5% CO₂ incubator at 37° C. for 24-72 h.
(3) The survival of tumor cells was observed by a fluorescence microscope, the dead tumor cells would lose the expression of green fluorescent proteins, and based on this, the in vitro killing efficiency of different 4SCAR-79b-CAR-T cells was evaluated. The results are shown in FIGS. 4A, 4B and 4C, where 79b-CART represents 4SCAR-79b-CAR-T. FIG. 4A is images showing in vitro killing of CD79b-positive tumor cell strains by different types of T cells at 24 h and 48 h (with a magnification factor of 50×), FIG. 4B is an image showing statistical results of remaining target cells quantified by flow cytometry after the killing of CD79b-positive tumor cell strains by different types of T cells at 24 h and 48 h, and FIG. 4C is an image showing statistical results of target cell death percentages after the killing of CD79b-positive tumor cell strains by different types of T cells at 24 h.

As can be seen from FIGS. 4A, 4B and 4C, compared with the CAR-T negative control group, the 4SCAR-79b-CAR-T experimental group (79b-CART) has a significant killing effect on the tumor cell strain, confirming that the CAR vector material can rapidly screen the effective CAR structure for subsequent clinical application.

### Example 6: Therapeutic Effect of Humanized 4SCAR-79b-CAR-T Cells

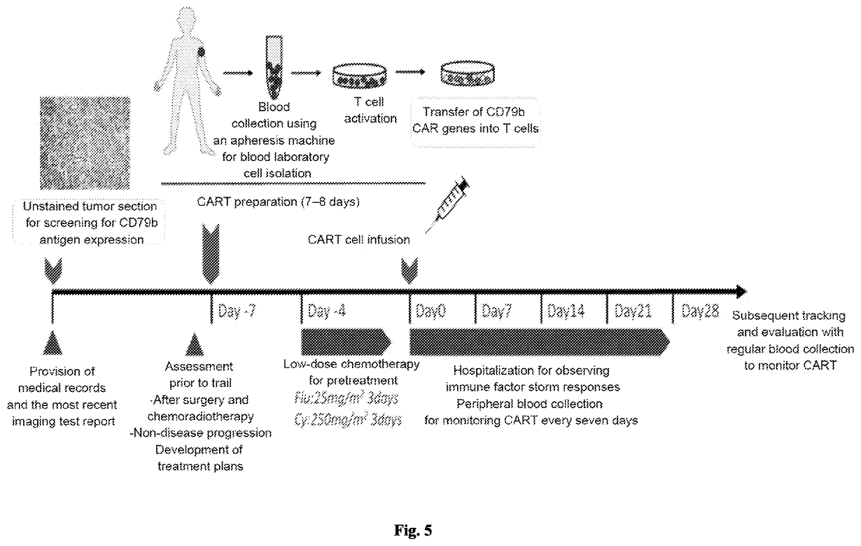

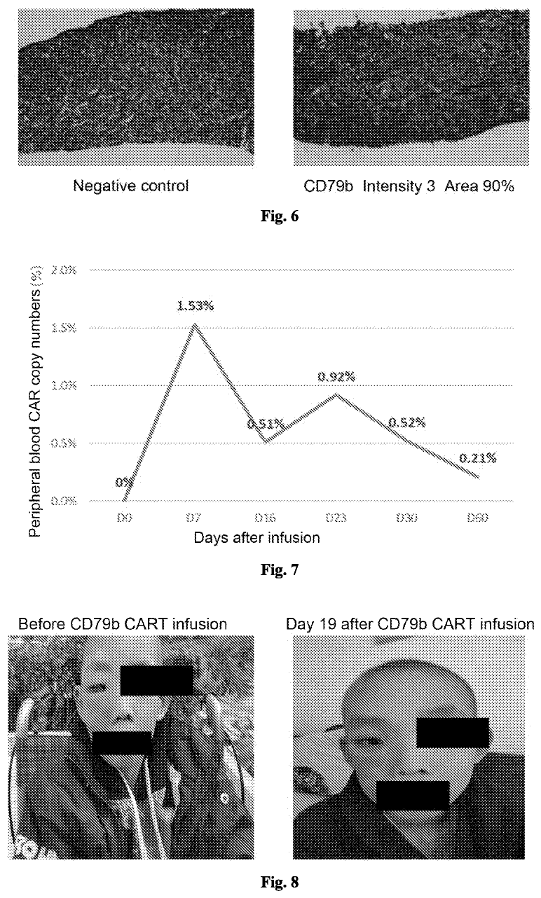

The patient in this example is from a partner children's hospital for clinical trial and treatment.
(1) The flowchart of 4SCAR-79b-CAR-T treatment for a B-cell tumor is shown in FIG. 5. The positive expression of CD79b in unstained tumor sections of the patient with diffuse large B-cell lymphoma (DLBCL) was confirmed by immunohistochemical staining. As shown in FIG. 6 which is an image showing immunohistochemical staining results of tumor sections of the patient with DLBCL (with a magnification factor of 20×), CD79b is highly expressed in DLBCL tumor tissues.
(2) The concentrate of white blood cells was collected from the patient. Peripheral mononuclear lymphocytes in the concentrate of white blood cells were separated by density gradient centrifugation with Ficoll, and T cells were screened by CD3 magnetic beads and activated by an anti-CD28 antibody. 1×10⁶ CAR-T cells were prepared per kilogram of the body weight for subsequent preparation of 4SCAR-79b-CAR-T.
(3) Before infusion, the patient was pretreated by low-dose chemotherapy. The preparative regimen was as follows: cyclophosphamide (250 mg/m²) for three days and fludarabine (25 mg/m²) for three days. The CAR-T infusion was conducted 24 h after the pretreatment, and the pretreatment was completed within three days.
(4) CAR-T cells were infused intravenously.
(5) After infusion, the doctor monitored the patient and evaluated the toxic reaction.
(6) After infusion, a small amount of peripheral blood was periodically collected from the patient, peripheral mononuclear lymphocytes were separated, and then genomic DNA (gDNA) was extracted. The CAR copy number in the peripheral blood was quantified by qPCR using specific primers. The results are shown in FIG. 7 which is a curve graph of in vivo CAR copy numbers detected in the DLBCL patient infused with 4SCAR-79b-CAR-T. As can be seen from FIG. 7, about 7 days after the patient was infused with CAR-T, the in vivo CAR-T value reaches a peak, and the CAR-T can be maintained in vivo for about 60 days without the occurrence of cytokine release syndrome (CRS).
(7) After 4SCAR-79b-CAR-T infusion, the size of the tumor on the face of the DLBCL patient was evaluated. The results are shown in FIG. 8 which shows the change of the tumor on the right face of the DLBCL patient before and after infusion with 4SCAR-79b-CAR-T. As can be seen from FIG. 8, 19 days after the patient was infused with 4SCAR-79b-CAR-T, the tumor on the right face shrinks, and the function of the right eye recovers.

In summary, the 4SCAR-79b chimeric antigen receptor of the present disclosure has better effects than other reported chimeric antigen receptors, has safety (no or low CRS reaction) and long-term efficacy, and indeed achieves better therapeutic effects for patients with B-cell tumors.

The applicant has stated that although the detailed method of the present application is described through the examples described above, the present application is not limited to the detailed method described above, which means that the implementation of the present application does not necessarily depend on the detailed method described above. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. A CD79b humanized antibody-based chimeric antigen receptor, comprising an antigen binding domain, a transmembrane domain, a co-stimulatory signaling domain, a CD3ζ signaling domain, and a self-destructing domain, which are sequentially connected in tandem;
wherein the antigen binding domain is a single-chain variable fragment (scFv) targeting a tumor surface antigen CD79b, and the amino acid sequence of the scFv targeting the tumor surface antigen CD79b is selected from:
an amino acid sequence shown in SEQ ID NO: 1; or an amino acid sequence which is formed by amino acid substitutions, additions or deletions to the amino acid sequence shown in SEQ ID NO: 1, wherein the amino acid sequence specifically binds to the chimeric antigen receptor and has functions of binding to CD79b and inducing T cell signaling.

2. The CD79b humanized antibody-based chimeric antigen receptor according to claim 1, wherein the amino acid sequence which is formed by amino acid substitutions, additions or deletions to the amino acid sequence shown in SEQ ID NO: 1 has at least 90% identity to the amino acid sequence shown in SEQ ID NO: 1 and preferably, is an amino acid sequence having at least 95% identity.

3. The CD79b humanized antibody-based chimeric antigen receptor according to claim 1, wherein the transmembrane domain is a CD28 transmembrane domain and/or a CD8a transmembrane domain;
preferably, the co-stimulatory signaling domain is a combination of a CD28 signaling domain and a CD27 signaling domain;
preferably, the CD28 transmembrane domain comprises a CD28 extracellular signaling domain and a CD28 cell membrane signaling domain; the co-stimulatory signaling domain comprises a CD28 signaling domain and a CD27 signaling domain; the amino acid sequence of the CD28 extracellular signaling domain, the CD28 cell membrane signaling domain, the CD28 signaling domain and the CD27 signaling domain together are shown in SEQ ID NO: 2;
preferably, the self-destructing domain comprises a cysteine protease 9 domain;
preferably, the amino acid sequence of the cysteine protease 9 domain is shown in SEQ ID NO: 7;
preferably, the self-destructing domain is connected in tandem with the CD3ζ signaling domain through a 2A sequence.

4. The CD79b humanized antibody-based chimeric antigen receptor according to claim 1, wherein the chimeric antigen receptor comprises a signal peptide, an antigen binding domain, a transmembrane domain, a co-stimulatory signaling domain, a CD3ζ signaling domain, a 2A sequence, and a self-destructing domain, which are sequentially connected in tandem;
preferably, the chimeric antigen receptor is formed by connecting in tandem a Secretory signal peptide, a CD79b antigen binding domain, a CD28 transmembrane domain and/or a CD8a transmembrane domain, a CD28 signaling domain, a CD27 signaling domain, a CD3ζ signaling domain, a 2A sequence, and a cysteine protease 9 domain;
preferably, the amino acid sequence of the chimeric antigen receptor is shown in SEQ ID NO: 10.

5. A nucleic acid molecule, encoding the CD79b humanized antibody-based chimeric antigen receptor according to claim 1.

6. A viral vector, comprising at least one copy of the nucleic acid molecule according to claim 5;
preferably, the viral vector is a lentiviral vector and/or a retroviral vector and preferably, is a lentiviral vector.

7. A recombinant lentivirus, prepared by a preparation method comprising the following step: co-transducing the viral vector according to claim 6 with packaging helper plasmids pNHP and pHEF-VSVG into a mammalian cell to obtain the recombinant lentivirus;
wherein preferably, the mammalian cell comprises a 293 cell, a 293 T cell or a TE671 cell.

8. A chimeric antigen receptor T cell, prepared by a preparation method comprising the following step: transducing the recombinant lentivirus according to claim 7 into a T cell for expression to obtain the chimeric antigen receptor T cell.

9. A composition, comprising the CD79b humanized antibody-based chimeric antigen receptor according to claim 1.

10. A method for treating a tumor, comprising administering the CD79b humanized antibody-based chimeric antigen receptor according to claim 1 to a subject in need thereof;
wherein preferably, the tumor is a tumor disease in which a CD79b specific antigen is expressed;
preferably, the tumor disease in which the CD79b specific antigen is expressed is a B-cell tumor.

11. The CD79b humanized antibody-based chimeric antigen receptor according to claim 1, wherein the amino acid sequence of the scFv targeting the tumor surface antigen CD79b comprise the amino acid sequence shown in SEQ ID NO: 1.


SEQUENCE LISTING (11 sequences)

SEQ ID NO: 1 - protein, 257 aa, synthetic construct
QVQLVESGPGVVQPGRSLRISCAVSGFSLSNYAVSWVRQPPGKGLEWLGIIYIETGTTWYANWAKGRLTISKDNSKNTVYLQMNSLRAEDTAMYYCASEPYEPYDDSNIYYGMDPWGQGTLVTVSSGSTSGSGKPGSSEGSTKGEIVMTQTPATLSVSAGERVTITCQASQSVVSGNYLAWYQQKPGQAPRLLIYSASTLASGVPARFSGSGYGTEFTFTISSVQSEDFAVYFCLGEFSCSSHDCNAFGQGTKLEIK

SEQ ID NO: 2 - protein, 167 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSQRRKYRSNKGESPVEPAEPCHYSCPREEEGSTIPIQEDYRKPEPACSP

SEQ ID NO: 3 - protein, 39 aa, synthetic construct
IEVMYPPPYLDNEKSNGTIIHVKGKHLCPSPLFPGPSKP

SEQ ID NO: 4 - protein, 27 aa, synthetic construct
FWVLVVVGGVLACYSLLVTVAFIIFWV

SEQ ID NO: 5 - protein, 41 aa, synthetic construct
RSKRSRLLHSDYMNMTPRRPGPTRKHYQPYAPPRDFAAYRS

SEQ ID NO: 6 - protein, 48 aa, synthetic construct
QRRKYRSNKGESPVEPAEPCHYSCPREEEGSTIPIQEDYRKPEPACSP

SEQ ID NO: 7 - protein, 401 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGAMVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 8 - protein, 22 aa, synthetic construct
MLLLVTSLLLCELPHPAFLLIP

SEQ ID NO: 9 - protein, 21 aa, synthetic construct
MALPVTALLLPLALLLHAARP

SEQ ID NO: 10 - protein, 996 aa, synthetic construct
MLLLVTSLLLCELPHPAFLLIPQVQLVESGPGVVQPGRSLRISCAVSGFSLSNYAVSWVRQPPGKGLEWLGIIYIETGTTWYANWAKGRLTISKDNSKNTVYLQMNSLRAEDTAMYYCASEPYEPYDDSNIYYGMDPWGQGTLVTVSSGSTSGSGKPGSSEGSTKGEIVMTQTPATLSVSAGERVTITCQASQSVVSGNYLAWYQQKPGQAPRLLIYSASTLASGVPARFSGSGYGTEFTFTISSVQSEDFAVYFCLGEFSCSSHDCNAFGQGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSQRRKYRSNKGESPVEPAEPCHYSCPREEEGSTIPIQEDYRKPEPACSPGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGAMVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 11 - protein, 5 aa, synthetic construct
GGGGS